(19) United States
(12) Patent Application Publication
(10) Pub. No.: US 2008/0262084 A1
(43) Pub. Date: Oct. 23, 2008

(54) Stable Emulsion Compositions for Intravenous Administration Having Preservatie Efficacy

(76) Inventors: Gautam Vinod Daftary, Mumbai (IN); Srikanth Annappa Pai, Mumbai (IN); Girish Narasimha Shanbhag, Mumbai (IN)

Correspondence Address: THORPE NORTH & WESTERN, LLP., P.O. Box 1219, SANDY, UT, 84091-1219, US

(21) Appl. No.: 11/662,750

(22) PCT Filed: Apr. 7, 2005
(86) PCT No.: PCT/IN05/00104

§ 371 Date: Mar. 7, 2008

(30) Foreign Application Priority Data
Sep. 13, 2004 (IN) 979/MUM/2004

(51) Int. Cl. A01N 37/02; A61K 31/05

(52) U.S. Cl. 514/552; 514/731

(57) ABSTRACT

Monoglycerides, especially Monolaurin, are used to protect intravenously administrable oil-in-water emulsion compositions against growth of E. coli, P. aeruginosa S. aureus and C. albicans. The compositions can be medicaments containing lipophilic drugs, especially Propofol, and/or total intravenous nutritional compositions.

### FIELD OF INVENTION

This invention relates to stable oil-in-water emulsion compositions for intravenous administration having preservative efficacy. It particularly relates to stable oil-in-water emulsion compositions of Propofol for intravenous administration, having preservative efficacy. Other embodiment of this invention relates to oil-in-water emulsion compositions having preservative efficacy of oils and fats for intravenous feeding. Another embodiment of this invention relates to oil-in-water emulsion compositions having preservative efficacy containing combination of lipophilic drugs and hydrophilic drugs. Yet another embodiment of this invention relates to oil-in-water emulsion compositions having preservative efficacy comprising intravenous fat emulsion compositions and hydrophilic/lipophilic drugs.

Compositions for intravenous administration need to satisfy more stringent requirements of safety than those prepared for other mode of administration such as oral dosage forms, dosage forms for external use etc.

### BACKGROUND AND PRIOR ART

A: Intravenous Propofol Emulsion Compositions:

Propofol (i.e. 2,6-Diisopropylphenol) is a well-known and widely used intravenous anaesthetic agent. Propofol is a hydrophobic, water-insoluble oil. To overcome the solubility problem, it must be incorporated with solubilising agents, surfactants, and/or solvents.

U.S. Pat. No. 5,637,625 (issued 10 Jun. 1997; corresponding to EP-A-07996616, published 24 Sep. 1997) discloses formulations of phospholipid-coated microdroplets of propofol devoid of fats and triglycerides providing chronic sedation over extended periods of time without fat overload. Being free of nutrients that support bacterial growth, these microdroplet formulations are bacteriostatic and bactericidal (e.g. self-sterilizing) and thus have extended shelf life. The following paragraphs from the U.S. Pat. No. 5,637,625 specification are reproduced for clear understanding.

. . . “The coating material of the propofol microdroplet can be chosen from the lipids described in my U.S. Pat. No. 4,725,442 (incorporated herein by reference) columns 5-7, particularly the phospholipids described in Class A, B and C. Additionally, the microdroplet can be coated by certain mono-glycerides capable of forming oriented monolayers and bilayers in the presence of decane (Benz et al. Biochim. Biophys. Acta 394:323-334, 1975). Examples of useful mono-glycerides include, but are not limited to, the following: 1-monopalmitoyl-(rac)-glycerol (onopalmitin); 1-monocaprylol-(rac)-glycerol (Monocaprylin); 1-monooleoyl-(rac)-glycerol (C18:1, cis-9) (Monoolein); 1-monostearyl-(rac)-glycerol (Monostearin)” . . . .

. . . “The phospholipid-coated microdroplets at about 0.1 μm diameter droplet of drug in the oil state, coated with a stabilizing monolayer of phospholipid are described in my earlier patents U.S. Pat. Nos. 4,622,219 and 4,725,442, the disclosures of which are hereby incorporated by reference. Microdroplet formulations have been made for many compounds including methoxyflurane, isoflurane and Vitamin E. The present invention provides a formulation of microdroplet propofol which allows the administration of propofol without the fat.” . . . .

The microdroplet composition described in this U.S. patent does not contain any fat, and therefore does not support any microbial growth. Also, the injection becomes painful beyond tolerance.

Propofol injections usually are made by diluting Propofol in oils and then formulated into oil-in-water type of emulsions. The compositions of the Propofol incorporated into the oily phase and made into oil-in-water emulsions for intravenous administration are termed hereafter “intravenous Propofol emulsion compositions.”

Intravenous fat emulsions used for total parenteral nutrition are termed hereafter “intravenous fat emulsion compositions”.

A Propofol/soybean oil emulsion has gained widespread use for induction and/or maintenance of anaesthesia, for maintenance of monitored anaesthesia care and for sedation in the Intensive Care Unit (ICU). It is advantageous in that it possesses both a rapid onset anaesthesia and a short recovery time.

However, the presence of vegetable oils and phospholipids makes the emulsion highly prone to the risk of microbial growth due to adventitious extrinsic contamination especially during long term use in patients undergoing ICU sedation.

Intravenous Propofol emulsion compositions are being increasingly used for sedation of seriously ill patients particularly in ICUs wherein it is continuously infused. There are nosocomial (i.e. hospital acquired) infections observed very often in ICU patients. Microbial contamination of total intravenous nutritional emulsion formulation supplements administered through infusion sets is recognized as one of the main reasons of nosocomial infection among ICU patients. Hence it is recommended that the intravenous administration sets are changed frequently, at least every 6 or 12 hours. Continuous infusion makes the product susceptible to microbial growth.

In order to reduce the risk of uncontrolled microbial growth, additions of various potential preservatives into intravenous Propofol emulsion compositions have been tried. Some of the potential agents found to cause instability of the emulsion. Other potential agents failed to provide the level of antimicrobial activity being sought. It is necessary to preserve the compositions with preservatives that would provide the required levels of antimicrobial activity at as low a concentration as possible in order to minimise the potential for physical instability and to minimise toxicity concerns.

EP-A-0814787 (published 7 Jan. 1998; corresponding to U.S. Pat. No. 5,714,520, issued 3 Feb. 1998) discloses an oil-in-water emulsion of Propofol containing an edetate as an antimicrobial agent. The amount of edentate is preferably no more than 0.1% by weight but is sufficient to prevent a no more than 10 fold increase in the growth of each of staphylococcus aureus (ATCC 6538) Eschericha coli (ATCC 8739), Pseudomonas aeruginosa (ATCC9027), and Candida albicans (ATCC 10231) for at least 24 hours as measured by a test wherein a washed suspension of each organism is added to a separate aliquot of said composition at approximately 50 colony-forming units per ml at a temperature in the range 20-25° C., incubated in that temperature range and tested for viable counts of said organism after 24 hours. The currently marketed formulation comprises 1% w/v Propofol, 10% w/v Soybean Oil, 1.2% w/v Egg Phosphatides as an emulsifier, 2.25% w/v Glycerol and 0.0055% w/v disodium edetate, Sodium hydroxide and Water for Injection.

Edetate has been shown to delay but not to prevent the onset of microbial growth in Propofol emulsions (see WO-A-00/24376, infra). Propofol emulsion compositions are required to be diluted up to 5 times (1:4) for long-term infusion. On dilution the edetate concentration gets reduced to 0.0011%. Edetate is found to be ineffective in preventing a no more than 10 fold increase in broad spectrum microbial growth at concentrations of 0.0025% and below (see U.S. Pat. No. 6,028,108; infra).

Edetate acting as a preservative in this formulation is a metal ion chelator that removes essential trace elements like zinc. This can be potentially dangerous to patients who are administered Propofol for a prolonged duration as it will cause deficiency of zinc in certain individuals. Even the manufacturer of this product recommends supplemental zinc therapy to overcome the untoward effects.

WO-A-99/39696 (published 12 Aug. 1999; corresponding to U.S. Pat. No. 6,469,069 issued 22 Oct. 2002) discloses an oil-in-water emulsion of Propofol containing a sulphite as an antimicrobial agent. The amount of sulphite preferably is in the range 0.0075% to 0.66% by weight and is sufficient to prevent a no more than 10 fold increase in the growth of each of staphylococcus aureus (ATCC 6538) Eschericha coli (ATCC 8739), Pseudomonas aeruginosa (ATCC9027), and Candida albicans (ATCC 10231) for at least 24 hours as measured by a test wherein a washed suspension of each organism is added to a separate aliquot of said composition at approximately 50 colony-forming units per ml and incubated at a temperature in the range 30-35° C. and tested for viable counts of said organism after 24 hours. The use of sulphite has two problems; viz. (a) stability of the emulsion is affected and (b) it is potentially toxic material at little higher dose level.

Reference is made to the water-immiscible solvent of the oil-in-water emulsion being a mono-, di-, or triglyceride. The preferred amount of solvent is 5 to 25% by weight.

Infusion of preferred compositions is accordance with WO-A-99/39696/U.S. Pat. No. 6,469,069 at a rate of 50 μg/kg/min for 24 hours will result in sulphite concentrations approaching the toxic levels. Further, the compositions may cause allergic reactions because of the sulphite molecule and the compositions have been reported to be physically and chemically unstable on exposure (see Han J et al International Journal of Pharmaceutics 2001, 215(1-2:207-220 & Baker M T et al Anesthesiology 2002, 97(5): 1162-1167).

U.S. Pat. No. 6,028,108 (issued 22 Feb. 2000; corresponding to WO-A-00/23050, published 27 Apr. 2000) discloses an oil-in-water emulsion of Propofol containing a pentetate as an antimicrobial agent. Preferably, the pentetate is present in an amount of 0.0005% to 0.1% by weight sufficient to prevent a no more than 10 fold increase in the growth of each of staphylococcus aureus (ATCC 6538) Eschericha coli (ATCC 8739), Pseudomonas aeruginosa (ATCC9027), and Candida albicans (ATCC 10231) for at least 24 hours after adventitious extrinsic contamination.

Pentetate, as used herein, refers to diethylene triamine pentaacetate or “DTPA”, and derivatives thereof. In general, suitable derivatives of DTPA are those salts having lower affinity for DTPA than calcium. Particular derivatives include but are not limited to calcium trisodium pentetate.

Pentetate acting as a preservative in this formulation is a metal ion chelator that removes cations like calcium, magnesium and zinc. This can be potentially dangerous to patients who are administered Propofol for a prolonged duration.

WO-A-00/24376 (published 4 May 2000; corresponding to U.S. Pat. No. 6,140,373 & U.S. Pat. No. 6,140,374, both issued 31 Oct. 2000) discloses an oil-in-water emulsion of Propofol containing an antimicrobial agent selected from (a) benzyl alcohol alone or, preferably, together with either sodium edetate or sodium benzoate and (b) benzethonium chloride. Preferably, the composition comprises Propofol 0.1-5.0% by wt.; vegetable oil, preferably soybean oil, 1-30% by wt; surfactant, preferably egg phosphatide, 0.2 to 2% by wt.; glycerol 2-3% by wt.; and antimicrobial agent selected from (i) benzyl alcohol 0.0175-0.9% by wt., (ii) benzyl alcohol 0.07-0.9% by wt and sodium edetate 0.005% by wt., (iii) benzethonium chloride 0.01% to 0.1% by wt. and, most preferably, (iv) beizyl alcohol 0.0175-0.9% by wt. and sodium benzoate 0.07% by wt.

Reference is made to the water-immiscible solvent of the oil-in-water emulsion being an ester of a medium or long chain fatty acid, exemplified as a mono-, di-, or triglyceride. The preferred amount of solvent is 10 to 20% by weight.

For long-term use, the antimicrobial agents such as benzyl alcohol and benzethonium chloride are not recommended as they are toxic.

WO-A-00/56364 (published 28 Sep. 2000; corresponding to U.S. Pat. No. 6,177,477, issued 23 Jan. 2001) discloses sterile pharmaceutical compositions for parenteral administration containing Propofol in an oil-in-water emulsion containing tromethamine (i.e. 2-amino-2-hydroxymethyl-1,3-propanediol) as an antimicrobial agent in an amount sufficient to prevent significant growth of microorganisms for at least 24 hours after adventitious extrinsic contamination. Preferably, the tromethamine is present in an amount of 0.15% to 0.25% by weight. The pH of the composition is highly alkaline and will degrade phospholipids and Propofol on long-term storage.

Further, tromethamine is known to cause extravasation at the site of injection and may cause tissue damage and also is reported to cause respiratory depression.

WO-A-00/59471 (published 12 Oct. 2000; corresponding to U.S. Pat. No. 6,100,302, issued 8 Aug. 2000) discloses intravenous anaesthetic Propofol emulsions having decreased levels of soybean oil, fats or triglycerides. The formulation preferably consists of phospholipid-coated microdroplets ranging from 160 to 200 nanometers in diameter. These microdroplets contain a sphere of Propofol dissolved in a solvent, such as vegetable oil, surrounded by a stabilizing layer of a phospholipid. It is reported that this formulation can safely provide sedation over extended periods of time and that the low oil concentration emulsion containing Propofol provides a stable oil-in-water emulsion and unexpectedly exhibits antimicrobial properties comparable to higher water immiscible solvent concentration emulsions containing preservatives.

Typically the emulsion comprises from 0.1 to 5%, by weight, preferably 1% to 2% by weight, of Propofol. The water-immiscible solvent, preferably soybean oil, is suitably present in an amount that is from 0.1 to 3% and more suitably from 1 to 3% by weight of the composition. However, the reduction in the oil content makes the injection more painful because of free Propofol in the aqueous phase.

WO-A-00/59475 (published 12 Oct. 2000; corresponding to U.S. Pat. No. 6,383,471 issued 7 May 2002) describes a pharmaceutical composition including a hydrophobic therapeutic agent having at least one ionizable functional group and a carrier. The carrier includes an ionizing agent capable of ionizing the functional group, a surfactant, and optionally solubilisers, triglycerides, and neutralizing agents. It also describes a method of preparing such compositions by providing a composition of an ionizable hydrophobic therapeutic agent, an ionizing agent, and a surfactant, and neutralizing a portion of the ionizing agent with a neutralizing agent. The compositions are particularly suitable for use in oral dosage forms and can be filled in capsules or made into a dosage form by coating on a particulate carrier. They also can be formulated as a solution, a cream, a lotion, an ointment, a suppository, a spray, an aerosol, a paste or a gel. Alternative dosage forms can be administered by routes selected from the group consisting of oral, parenteral, topical, transdermal, ocular, pulmonary, vaginal, rectal and transmucosal.

Listed surfactants for use in the compositions include monoglycerides with specific reference to Monolaurin and listed therapeutic agents include Propofol but there is no exemplification of any monoglyceride-containing or Propofol-containing composition.

It is believed that of the prior art compositions discussed above, only two products, one with edetate and another with metabisulphite are commercially available. Thus a need exists to develop an intravenous Propofol emulsion composition with improved preservative efficacy.

B: Intravenous Fat Emulsion Compositions:

Intravenous fat emulsion compositions containing emulsified vegetable oils have been in clinical use for nearly forty years. These were originally introduced to provide a source of calories for patients unable to ingest food. The total intravenous nutrition supplements are oil-in-water type emulsions. These intravenous oil feeding emulsion compositions are continuously improved with the advances in the nutritional requirements of the patients.

JP-A-58-230918 (acknowledged in U.S. Pat. No. 5,874,470 infra) describes an emulsion containing eicosapentaenoic acid for oral and non-oral use. Said emulsion contains from 1 to 40% w/v of eicosapentaenoic acid or, preferably, its methyl or ethyl ester; from 1 to 30% w/v of a vegetable oil, preferably soybean oil; from 0.01 to 30% w/v of alpha-tocopherol; and, as emulsifiers, from 0.1 to 5% w/v of a phospholipid, preferably from egg yolk and/or soybean; and from 0.1 to 10% w/v of a non-ionic synthetic emulsifier.

DE-A-3409793 (published 20 Sep. 1984; corresponding to U.S. Pat. No. 5,034,414, issued 23 Jul. 1991) discloses a nutritional liquid emulsion for transfusion comprising at least one C₂₀₋₂₂ fatty acid or ester thereof or a mixture thereof, a vegetable oil, an emulsifier and water. It is reported to possess antithrombic and antiarteriosclerotic activity as well as being nutritionally valuable. Preferably, the composition comprises 5 to 20% w/v of the fatty acid(s) or ester(s), 1 to 19% w/v of a vegetable oil, 1 to 2% w/v of an emulsifier, and 1 to % w/v of an emulsion stabilizer. The vegetable oil preferably is soybean oil and/or safflower oil, and the emulsifier preferably is egg yolk or soybean lecithin.

EP-A-0145873 (published 26 Jun. 1985) discloses a transfusion emulsion comprising fat, an emulsifier and water and intended as a nutritional supplement. The fat phase consists of from 10 to 50% w/v of an alpha-linolenic acid ester, preferably the triglyceride or ethyl ester. The balance of the fat can be a vegetable oil, preferably safflower oil or soybean oil

EP-A-0311091 (published 12 Apr. 1989; corresponding to U.S. Pat. No. 5,874,470, issued 23 Feb. 1999) discloses isotonic fat emulsions incorporating omega-3-fatty acids, omega-6-fatty acids, and medium-chain triglycerides. The emulsions are intended for parenteral application in post-aggression metabolism, in cases of chronic inflammatory diseases, and in neonatology and pediatrics and can provide a liver protective effect. Due to the combination of omega-3-fatty acids and/or their physiologically acceptable esters with medium-chain-triglycerides, the medium-chain triglycerides are preferred to be oxidized in the organism, and the omega-3 fatty acids are protected from rapid oxidation, so that they are available to a higher extent for the formation of triply unsaturated eicosanoids. The emulsions have a total fat content of 5 to 30% and an emulsifier content of 5 to 12% of the fat content. The emulsifier preferably is a phospholipid, glycerol can be used as isotonic agent and the preferred pH of the emulsion is 6-9.

There is no reference to the presence of antimicrobial agents; the only preservatives mentioned are antioxidants for the omega-3 fatty acid components.

None of such total intravenous nutritional formulation supplements are protected from possible microbial growth arising from extraneous contamination and leading to infections. Thus there is a need to improve such products and overcome the disadvantages of the intravenous fat emulsion compositions and particularly commercially available marketed formulations.

### OBJECT OF INVENTION

The main object of the present invention is to provide a sterile, stable pharmaceutical oil-in-water emulsion for intravenous administration that does not support significant microbial growth in (a) compositions of lipophilic drugs, especially Propofol, for intravenous administration (b) compositions of oils and fats for intravenous feeding and (c) compositions of lipophilic drugs and hydrophilic drugs and/or oils and fats for intravenous feeding.

More particularly the object of the present invention is to provide oil-in-water emulsion compositions having preservative efficacy to the extent that there will be no more than 10 fold increase for at least 24 hours in growth of each of Pseudomonas aeruginosa, Escherichia coli, Staphylococcus aureus and Candida albicans, after adventitious extrinsic contamination.

### SUMMARY OF THE INVENTION

In accordance with one aspect of the present invention, there is provided a sterile oil-in-water emulsion composition for intravenous administration comprising, as an antimicrobial preservative, a monoglyceride, preferably Monolaurin. Preferably the monoglyceride is present in an amount sufficient to prevent a no more than 10 fold increase in the growth of microbial cultures each of Candida albicans ATCC 10231, Pseudomonas aeruginosa ATCC 9027, Escherichia coli ATCC 8739 and Staphylococcus aureus ATCC 6538 for at least 24 hours after adventitious extrinsic contamination. The microbial growth is as measured by a test wherein a washed suspension of each organism is added to a separate aliquot of said composition at approximately 50 colony forming units per ml and incubated at a temperature in the range of 20-25° C. for culture of Candida albicans and at a temperature in the range of 30-35° C. for the remaining cultures and are tested for viable counts of said organisms after 24 hours and wherein the said amount of monoglyceride is no more than the antimicrobial equivalent against said cultures obtained with a composition containing 1.5% w/v Monolaurin.

The monoglycerides used in the intravenously administrable composition is preferably Monolaurin.

### DETAILED DESCRIPTION OF THE INVENTION

The amount of monoglyceride in the intravenously administrable composition typically is the antimicrobial equivalent against said cultures obtained with a composition containing up to 1% w/v Monolaurin, preferably up to 0.5% w/v Monolaurin, and more preferably up to 0.1% w/v Monolaurin.

In one embodiment, the intravenously administrable composition of the invention is for total parenteral nutrition and in another embodiment it is a medicament comprising a lipophilic drug, especially Propofol.

The content of lipophilic drug typically is from 0.001% w/v to 10% w/v of the composition, preferably from 0.01% to 5% w/v, and more preferably from 0.1% to 2% w/v.

Typically, the ratio of monoglyceride (calculated as Monolaurin) to lipophilic drug is from 1:0.01 to 1:5000 by weight, preferably from 1:0.2 to 1:1000 by weight, more preferably 1:4 to 1:200 by weight, and especially 1:20 to 1:100 by weight.

Usually the intravenously administrable composition of the invention will comprise at least one triglyceride oil and at least one phosphatide.

Preferably, the at least one triglyceride oil is selected from natural vegetable oils and synthetic MCT (medium-chain triglycerides) oil and the content of the triglyceride oil(s) is not more than 30% w/v of the composition, more preferably from 5% w/v to 20% w/v, and especially about 10% w/v or about 20% w/v. The preferred triglyceride oil is soybean oil.

Preferably, the at least one phosphatide is selected from purified egg lecithin and purified soya lecithin and the content of the phosphatide(s) is from 0.1% w/v to 3% w/v of the composition, especially about 1.2% w/v.

Usually the intravenously administrable composition of the invention will comprise at least one isotonic agent, preferably glycerin, and the composition is isotonic with blood.

Preferably, the intravenously administrable composition of the invention has a pH of between 6 and 8.5, conveniently adjusted by the presence of a relevant amount of sodium hydroxide.

In a second aspect, the present invention provides the use of a monoglyceride as an antimicrobial agent in a sterile oil-in-water emulsion composition for intravenous administration. In this aspect, the monoglyceride and/or other components of the intravenous administration composition can be as described above in connection with the first aspect.

In another aspect, the present invention provides a process of preparing an lipophilic drug-containing intravenously administrable composition of the invention comprising the steps of:
i) dissolving monoglyceride and the lipophilic drug in triglyceride oil maintained at elevated temperature;
ii) adding and dissolving phosphatide in the solution prepared in step i);
iii) preparing an aqueous phase by dissolving glycerin and sodium hydroxide in water and then heating the aqueous phase;
iv) adding the solution of step ii) to the aqueous phase obtained at step iii) under stirring to produce a coarse emulsion; and
v) homogenizing the coarse emulsion obtained at step iv)

In a further aspect, the present invention provides a process of preparing an intravenously administrable composition of the invention comprising the steps of:
i) dissolving monoglyceride and, if present, the lipophilic drug in triglyceride oil maintained at elevated temperature;
ii) preparing an aqueous phase by dissolving glycerin and sodium hydroxide in water and then heating the aqueous phase;
iii) adding and dispersing phosphatide in the aqueous phase prepared in step ii);
iv) adding the solution of step i) to the aqueous phase obtained at step iii) under stirring to produce a coarse emulsion; and
v) homogenizing the coarse emulsion obtained at step iv)

In the aforementioned process aspects, it is preferred that:

said homogenization is to an average globule size of less than 500 nanometers;

the homogenized composition is filtered;

the resultant filtrate is filled into containers, followed by nitrogen blanketing and the filled containers sealed; and

the sealed containers filled with the said filtrate sterilised by autoclaving.

A: Intravenous Propofol Emulsion Compositions:

In accordance with a preferred embodiment of the present invention, there is provided a sterile pharmaceutical oil-in-water emulsion composition for intravenous administration comprising
(i) Propofol;
(ii) one or more triglyceride oil-natural such as vegetable oils or synthetic such as MCT oil;
(iii) one or more naturally occurring phosphatides such as purified egg lecithin, soya lecithin;
(iv) isotonic agent(s) such as glycerin;
(v) Monolaurin in an amount sufficient to prevent a no more than 10 fold increase in the growth of microbial cultures each of Candida albicans ATCC 10231, Pseudomonas aeruginosa ATCC 9027, Escherichia coli ATCC 8739 and Staphylococcus aureus ATCC 6538 for at least 24 hours as measured by a test wherein a washed suspension of each organism is added to a separate aliquot of said composition at approximately 50 colony forming units per ml and incubated at a temperature in the range of 20-25° C. for culture of Candida albicans and at a temperature in the range of 30-35° C. for the remaining cultures and are tested for viable counts of said organisms after 24 hours and wherein the said amount of Monolaurin being no more than 1% w/v of the said composition.

One preferred composition of this embodiment comprises

Propofol about 1% w/v,

Soybean oil about 10% w/v,

Purified egg lecithin about 1.2% w/v,

Glycerin about 2.25% w/v,

Monolaurin about 0.05% w/v,

Sodium hydroxide sufficient to bring the pH between 6 and 8.5 and

Water for Injection to make up to 100% by volume.

Another preferred composition of this embodiment comprises

Propofol about 2% w/v,

Soybean oil about 10% w/v,

Purified egg lecithin about 1.2% w/v,

Glycerin about 2.25% w/v,

Monolaurin about 0.05% w/v,

Sodium hydroxide sufficient to bring the pH between 6 and 8.5 and

Water for Injection to make up to 100% by volume.

Another preferred composition of this embodiment comprises

Propofol about 1% w/v,

Soybean oil about 10% w/v,

Purified egg lecithin about 1.2% w/v,

Glycerin about 2.25% w/v,

Monolaurin about 0.01% w/v,

Sodium hydroxide sufficient to bring the pH between 6 and 8.5 and

Water for Injection to make up to 100% by volume.

As explained above, Propofol emulsion compositions are prone to microbial contamination and hence they need to be preserved with preservatives so that the product does not support the growth of microorganisms in case of adventitious extrinsic contamination. As all antimicrobial agents are toxic, for maximum protection of the patients, the concentration of the preservative is required to be kept at a minimum level to achieve required inhibition of the growth of the organisms.

Of the prior art patents cited above, five of them are oil-in-water emulsions and use preservatives. The requirement of the preservatives in these compositions is to provide preservative efficacy to the extent that there will be no more than 10 fold increase for at least 24 hours in growth of each of Pseudomonas aeruginosa, Escherichia coli, Staphylococcus aureus and Candida albicans, after adventitious extrinsic contamination and the preservative used will be safe.

Edetate, Pentetate and Metabisulphite used in acidic pH fulfil the above test. However, Edetate and Pentetate deplete zinc from the body and Metabisulphite is harmful in the long run.

Benzyl alcohol, sodium ethylene diamine tetraacetate; benzethonium chloride and sodium benzoate are broadly classed as antimicrobial agents which delay onset or retard rate of growth to less than 1 logarithmic increase over a 24 hour period as compared to an unpreserved formulation. However, they are toxic in the long run.

Tromethamine used as a preservative in sterile intravenous fat emulsions is in an amount sufficient to prevent an at least ten fold increase in growth of microorganisms for at least 24 hours after extrinsic contamination. However, it causes extravasation at the site of injection and may cause tissue damage and also respiratory depression.

Propofol:

The Propofol compositions of the present invention typically comprise 0.01% to 5% w/v of Propofol. Preferably the compositions comprise 0.1% to 2% w/v of Propofol. More preferably the compositions comprise about 1% and about 2% w/v of Propofol.

Triglyceride:

The triglyceride oil(s) content in preferred compositions of the invention is up to 30% w/v of the composition, preferably in the range of 5% to 20% w/v of the composition, more preferably about 10% w/v and about 20% w/v of the composition.

Triglyceride oil suitable for the compositions of present invention include natural oils such as vegetable oils, or synthetic oils such as MCT oil. Typically, the natural oil will be a vegetable oil and preferably is selected from the group consisting of Soybean oil, Sunflower oil, Safflower oil, Arachis oil, Cottonseed oil. The synthetic oil typically is manufactured from a vegetable oil which is chemically and/or physically modified and/or purified. MCT oil is a typical example of synthetic oil and is obtained from the fixed oil extracted from the hard, dried fraction of the endosperm of Cocos nucifera L. Hydrolysis of the fixed oil followed by distillation yields the required fatty acids, which are then re-esterified to produce MCT oil (Medium-chain Triglycerides). The present invention may also comprise any combination of one or more vegetable oils and/or synthetic oils. Soybean oil is the preferred natural vegetable oil used in the compositions of the present invention.

Phosphatide:

In the preferred compositions of present invention natural phosphatide is present in the range of 0.1% to 3% w/v, more preferably in the range of 0.6% to 1.5% w/v and most preferably about 1.2% w/v of the composition.

In the oil-in water emulsion compositions of the present invention natural phosphatide is used as an emulsifier for stabilization of the oil-in-water emulsion. The preferred natural phosphatide used is either purified egg lecithin or purified soya lecithin or a mixture thereof. More preferably the natural phosphatide used is purified egg lecithin.

In the present invention, it is preferred no emulsifiers other than phosphatides are used.

Phosphatides are well known for forming liposomes when hydrated with aqueous media and are used in the present invention as emulsifier and for stabilizing the emulsion. They are not used in the present forming liposomal compositions.

Isotonic Agents:

The composition of the present invention preferably is isotonic with blood by incorporating a suitable tonicity modifying agent such as Glycerin, Dextrose, or Mannitol. Glycerin is the preferred tonicity modifying agent.

Monoglycerides:

Fatty acid esters of the alcohol glycerol are called acylglycerols or glycerides. They are the major components of depot, or storage, fats in plant and animal cells, especially in the adipose cells of vertebrates. When one of the hydroxyl group of glycerol is esterified with fatty acids, it is called monoacylglycerols or monoglycerides. The saturated fatty acids include caproic acid, caprylic acid, capric acid, lauric acid, myristic acid, palmitic acid, stearic acid, arachidic acid, lignoceric acid. The unsaturated fatty acids include palmitoleic acid, oleic acid, linoleic acid, linolenic acid, arachidonic acid. The preferred monoglyceride is Monolaurin.

Monolaurin:

U.S. Pat. No. 5,714,520 (supra) discloses that for effectiveness, the antimicrobial properties of any preservatives have to be exerted in the aqueous phase. A preservative with lipophilic properties incorporated at typical usage levels would not be effective as although there would some partitioning between the phases, the concentration in the aqueous phase is insufficient to exert preservative effect. Increasing the overall quantity of such a preservative would result in unacceptably high levels of preservative in the lipid layer leading to toxicity problems at least.

U.S. Pat. No. 6,469,069 (supra) discloses that the preservative should be soluble in the aqueous phase and does not partition into the organic phase.

Contrary to the teachings of both U.S. Pat. No. 5,714,520 and U.S. Pat. No. 6,469,069, it has surprisingly been found that the monoglyceride of lauric acid (Monolaurin) which has no aqueous solubility is effective as a preservative in a concentration as low as 0.01%.

Food grade materials as preservatives in food, cosmetics and pharmaceutical preparations employing a combination of monoglycerides such as monolaurin, and medium chain fatty acids including caproic, caprylic fatty acids have been described in EP-A-0244144 (published 4 Nov. 1987; corresponding to U.S. Pat. No. 6,638,978, issued 28 Oct. 2003) and references therein. However, these compositions are not relevant for preserving oil-in-water emulsion compositions for intravenous administration.

Monolaurin as a preservative in this system provides preservative efficacy to the extent that there will be no more than 10 fold increase for at least 24 hours in growth of each of Pseudomonas aeruginosa, Escherichia coli, Staphylococcus aureus and Candida albicans, after adventitious extrinsic contamination and it is toxicologically safe.

Monolaurin as used in this application refers to all pharmaceutically acceptable glyceryl esters of lauric acid having molecular formula C₁₅H₃₀O₄ and a molecular weight of about 274.4. Commercially available Monolaurin is also known by other names such as “rac-1-Lauroylglycerol”, “1-Monododecanoyl-rac-glycerol”, “1-Monolauroyl-rac-glycerol”, “rac-Glycerol 1-laurate”, and “DL-α-Laurin”.

A mixture of 1 and 2 monoglycerides, or 2-monoglycerides of lauric acid are also Monolaurins. Monolaurin may contain some diglycerides of lauric acid. The purity of Monolaurin is not critical, it should be rich in C₁₂ (lauric) fatty acid but presence of some amounts of C₁₀, C₁₄ etc fatty acids are acceptable.

Monolaurin exhibits polymorphism. α form, β′ form and β form have been reported to have melting points of 44° C., 59.5° C. and 63° C. respectively.

The nature of Monolaurin used in this invention is not critical as long as it fulfils the requirements of preventing significant growth of microorganisms for at least 24 hours in the event of adventitious extrinsic contamination as described above. The requirements of the quantities may, to some extent, depend on the nature of the Monolaurin used.

Monolaurin is insoluble in aqueous media but is highly soluble in the so-called fat solvents such as chloroform, benzene, ethanol, or acetone.

In rats when Monolaurin is administered orally, the LD₅₀ has been reported to be about 53,000 mg/kg body weight.

It will be apparent to one skilled in the pharmaceutical arts that other monoglycerides such as Monostearin, Monopalmitin, Monocaprylin, Monoolein etc or mixture thereof may be used along with Monolaurin and that their concentration used preferably will be sufficient to prevent a no more than 10-fold increase in the growth of microbial cultures as described earlier. It will also be apparent to one skilled in the pharmaceutical arts that ethoxylated or propoxylated monoglycerides may be used to prevent a no more than 10-fold increase in the growth of microbial cultures as described earlier.

HLB value of Monolaurin is less than 10 and therefore it is suitable as an emulsifier or solubiliser only for making water-in-oil emulsions and not oil-in-water emulsions. It is used in small quantities in the present invention as a preservative and not as an emulsifier or as a solubiliser.

Compositions:

Typically Monolaurin will be present in the composition of the present invention in a concentration range of 0.001% to 1.5% w/v. Preferably Monolaurin is present in the range of 0.01% to 1% W/v, more preferably 0.01% to 0.5% w/v. The most preferred concentration of the Monolaurin is between 0.01% w/v and 0.1% w/v of the composition.

The compositions of the present invention can also be made as a concentrate containing higher quantities of lipophilic drugs and Monolaurin and appropriately diluted at the time of administration, for example emulsion concentrate containing higher quantities of Propofol and Monolaurin can be made and diluted appropriately at the time of administration. The weight ratio of Monolaurin to Propofol in such compositions is from 1:0.01 to 1:5000 by weight. Preferably it is from 1:0.2 to 1:1000 by weight, more preferably it is from 1:4 to 1:200 by weight and most preferably it is from 1:20 to 1:100 by weight.

The pH of the composition of the present invention usually is between 6 to 8.5 and may be adjusted as required using an alkali for example Sodium hydroxide.

A typical oil-in-water emulsion composition of the present invention comprises

Propofol about 1% w/v

soybean oil about 10% w/v

purified egg lecithin about 1.2% w/v

Glycerin about 2.25% w/v

Monolaurin about 0.01% w/v

Sodium hydroxide q.s. to adjust to required pH

Water to 100%.

In another composition, the Propofol is about 2% w/v

The Propofol-containing compositions of the invention can be prepared by a process comprising the steps of
i) dissolving Monolaurin and Propofol in triglyceride oil, preferably soybean oil, maintained at about 75° C.;
ii) adding and dissolving the emulsifier Purified egg lecithin in the solution of Propofol prepared in step i);
iii) preparing an aqueous phase by dissolving glycerin and sodium hydroxide in water and then heating the aqueous phase to about 70° C.;
iv) adding the Propofol solution of step ii) to Aqueous Phase obtained at step iii) under stirring to produce a coarse emulsion;
v) homogenizing the coarse emulsion obtained at step iv) to an average globule size of less than 500 nanometers;
vi) filtering the said composition obtained at the end of step v);
vii) filling the said filtrate obtained at the end of step vi) in containers such as vials, ampoules, followed by nitrogen blanketing and sealing the filled containers;
viii) sterilising the sealed containers filled with the said filtrate by autoclaving.

Another process of preparing the Propofol-containing compositions of the invention comprising the steps of
i) dissolving Monolaurin and Propofol in triglyceride oil, preferably soybean oil, maintained at about 75° C.;
ii) preparing an aqueous phase by dissolving glycerin and sodium hydroxide in water and then heating the aqueous phase to about 70° C.;
iii) adding and dispersing the emulsifier Purified egg lecithin in the aqueous phase prepared in step ii);
iv) adding the Propofol solution of step i) to Aqueous Phase obtained at step iii) under stirring to produce a coarse emulsion;
v) homogenizing the coarse emulsion obtained at step iv) to an average globule size of less than 500 nanometers;
vi) filtering the said composition obtained at the end of step v);
vii) filling the said filtrate obtained at the end of step vi) in containers such as vials, followed by nitrogen blanketing and sealing the filled containers;
viii) sterilising the sealed containers filled with the said filtrate by autoclaving.

Both the above processes can be followed for preparing compositions containing other lipophilic drugs and also wherein Propofol is replaced with another lipophilic drug.

B: Intravenous Fat Emulsion Compositions:

Another embodiment of the present invention is similar to the Propofol embodiment (supra) except that it does not contain Propofol.

This embodiment provides an intravenous fat emulsion for intravenous administration for nutrition purpose comprising one or more triglyceride oils-natural such as vegetable oils or synthetic such as MCT oil; one or more naturally occurring phosphatides such as purified egg lecithin or soya lecithin; and isotonic agent(s) such as glycerin; and Monolaurin in an amount sufficient to prevent a no more than 10 fold increase in the growth of microbial cultures each of Candida albicans ATCC 10231, Pseudomonas aeruginosa ATCC 9027, Escherichia coli ATCC 8739 and Staphylococcus aureus ATCC 6538 for at least 24 hours as measured by a test wherein a washed suspension of each organism is added to a separate aliquot of said composition at approximately 50 colony forming units per nil and incubated at a temperature in the range of 20-25° C. for culture of Candida albicans and at a temperature in the range of 30-35° C. for the remaining cultures and are tested for viable counts of said organisms after 24 hours and wherein the said amount of Monolaurin being no more than 1% w/v of the said composition. Other monoglycerides specified in the earlier embodiment could also be used replacing Monolaurin, preferably in an amount sufficient to prevent a no more than 10-fold increase in the growth of microbial cultures of the said organisms. Monolaurin is the preferred monoglyceride and other monoglycerides could be used in combination with Monolaurin in an amount sufficient to prevent a no more than 10-fold increase in the growth of microbial cultures of the said organisms.

Triglyceride oils are used as a source of providing calorie requirements to patients for whom oral nutrition is not possible.

Any edible grade oil or fat or a mixture thereof are used. Some of them in addition also contain a blend of other oils with certain amounts of polyunsaturated fatty acid (PUFA), monounsaturated fatty acid (MUFA) and omega-3 fatty acid.

Accordingly to a preferred embodiment, the sterile pharmaceutical oil-in-water fat emulsion composition for intravenous administration comprises
(i) one or more triglyceride oils-natural such as vegetable oils or synthetic such as MCT oil;
(ii) one or more naturally occurring phosphatides such as purified egg lecithin, soya lecithin;
(iii) isotonic agent(s) such as glycerin;
(iv) Monolaurin in an amount sufficient to prevent a no more than 10 fold increase in the growth of microbial cultures each of Candida albicans ATCC 10231, Pseudomonas aeruginosa ATCC 9027, Escherichia coli ATCC 8739 and Staphylococcus aureus ATCC 6538 for at least 24 hours as measured by a test wherein a washed suspension of each organism is added to a separate aliquot of said composition at approximately 50 colony forming units per ml and incubated at a temperature in the range of 20-25° C. for culture of Candida albicans and at a temperature in the range of 30-35° C. for the remaining cultures and are tested for viable counts of said organisms after 24 hours and wherein the said amount of Monolaurin being no more than 1% w/v of the said composition.

Preferences set forth above in connection the Propofol-containing embodiment apply also to this embodiment.

Preferably, Monolaurin will be present in the fat emulsion composition of the present invention in a concentration range of 0.001% to 1% w/v of the composition.

The preferred fat emulsion compositions can be prepared by a process comprising the steps of
i) dissolving Monolaurin in triglyceride oil, preferably soybean oil, maintained at about 75° C.;
ii) preparing an aqueous phase by dissolving glycerin and sodium hydroxide in water and then heating the aqueous phase to about 70° C.;
iii) adding and dispersing the emulsifier Purified egg lecithin in the aqueous phase prepared in step ii);
iv) adding the Monolaurin solution of step i) to Aqueous Phase obtained at step iii) under stirring to produce a coarse emulsion;
v) homogenizing the coarse emulsion obtained at step iv) to an average globule size of less than 500 nanometers;
vi) filtering the said composition obtained at the end of step v);
vii) filling the said filtrate obtained at the end of step vi) in containers such as vials, followed by nitrogen blanketing and sealing the filled containers;
viii) sterilising the sealed containers filled with the said filtrate by autoclaving.

C: Intravenous Lipophilic Drug Emulsion Compositions:

In another embodiment of the invention, any other lipophilic drug replaces the Propofol of the Propofol-containing embodiment.

A number of lipophilic drugs belonging to different groups such as steroids, antifungal agents, anesthetics, anticancer agents, psychotropic drugs, prostaglandins, antibiotics, fat-soluble vitamins may be incorporated in the triglyceride oil, emulsified and advantageously administered as an oil-n-water emulsion.

Some of the drugs that could be incorporated into intravenous emulsion compositions include for instance progesterone, hydrocortisone, prednisolone, betamethasone, itraconazole, clotrimazole, amphotericin B, propofol, benzocaine, lignocaine, paclitaxel, melphalan, lomustine, phenobarbitone, diazepam, alprostadil, carboprost, dinoprostone, misoprostol, nifepristone, clarithromycin, erythromycin, chloramphenicol, digoxin, vitamin A, vitamin E.

Accordingly, the present invention also provides therapeutic oil-in-water emulsion compositions comprising lipophilic pharmaceutical materials which further comprises an amount of monoglyceride, preferably Monolaurin, in a concentration sufficient to prevent significant growth of microorganisms for at least 24 hours in the event of adventitious extrinsic contamination.

Preferences set forth above in connection the Propofol-containing embodiment apply also to this embodiment.

D: Combination of Lipophilic and Hydrophilic Drug Containing emulsion Composition:

In another embodiment of the invention, combination of lipophilic drugs and hydrophilic drugs are also formulated with monoglyceride, preferably Monolaurin.

Preferences set forth above in connection the lipophilic drug emulsion compositions also apply to this embodiment. The hydrophilic drugs for instance include Ondansetron hydrochloride, diltiazem hydrochloride, frusemide, hydrochlorothiazide, lignocaine hydrochloride.

E: Combination of Drugs with Intravenous Fat Emulsion Composition:

In another embodiment of the invention, the composition comprises intravenous nutritional fat emulsion compositions and hydrophilic/lipophilic drugs.

In these compositions, the nutritional requirements and the drug requirements are provided simultaneously as per the need of the patients. For example such a composition when administered intravenously fulfils the need of sedation as well as nutrition for a patient after surgery.

Accordingly, an intravenously administrable composition with Monolaurin can also be prepared wherein the oil phase comprises lipophilic drugs and/or nutritive oils, and aqueous phase comprises hydrophilic drugs and/or water soluble nutrients.

Preferences set forth above in connection the fat emulsion compositions also apply to this embodiment.

Further, an intravenously administrable composition containing Monolaurin can also be prepared wherein the aqueous phase comprises hydrophilic drugs and/or water soluble nutrients.

Testing of Preservative Efficacy

The preservative efficacy of the compositions of the present invention were tested wherein a washed suspension of each of standard strains of Candida albicans ATCC 10231, Pseudomonas aeruginosa ATCC 9027, Escherichia coli ATCC 8739 and Staphylococcus aureus ATCC 6538, is added to a separate aliquot of said composition at approximately 50 to 250 colony forming units per ml. The said aliquots were incubated at a temperature of 20-25° C. for fungal culture and 30 to 35° C. for bacterial culture as recommended under “Antimicrobial effectiveness testing” in United States Pharmacopeia, Chapter 51 (U.S.P. <51>). The compositions capable of preventing a no more than 10 fold increase in growth of each of the said organisms for at least 24 hours after inoculation were concluded to meet the criteria of the objective of the invention.

### EXAMPLES

The invention will now be illustrated by way of Examples. The Examples are by way of illustration only and in no way restrict the scope of the invention.

### Materials and Equipment Used in the Examples

Propofol complies with The European Pharmacopoeia (Ph.Eur.) specifications,

Glycerin, Sodium hydroxide, Water for Injection complies with Indian Pharmacopoeia (I.P.) specifications.

Soya oil (Soybean oil) complies with U.S.P. specifications.

Purified egg lecithin (referred to as Egg lecithin in examples) is manufactured by M/s.Lipoid.

Monolaurin is a racemic mixture obtained from Sigma

High speed mixing was done using a laboratory Remi stirrer. Emulsions were homogenised using high pressure APV homogenizer.

### Examples I-IV

### Propofol Oil-in-Water Emulsion Compositions Containing Preservative Monolaurin

Compositions of Examples I-IV as given in Table 1

TABLE 1
Propofol oil-in-water emulsion compositions

| | | | | | | | Qty/100 ml | |
|---|---|---|---|---|---|---|---|---|
| Examples | I | | II | | III | | IV | |
| Propofol | 1 | g | 1 | g | 1 | g | 2 | g |
| Monolaurin | 0.2 | g | 0.05 | g | 0.01 | g | 0.05 | g |
| Soya Oil | 10 | g | 10 | g | 10 | g | 10 | g |
| Egg lecithin | 1.2 | g | 1.2 | g | 1.2 | g | 1.2 | g |
| Glycerin | 2.25 | g | 2.25 | g | 2.25 | g | 2.25 | g |
| Sodium | q.s. | | q.s. | | q.s. | | q.s. | |
| hydroxide | | | | | | | | |
| (0.1 N) | | | | | | | | |

| | | | | |
|---|---|---|---|---|
| Water for | q.s. to | q.s. to | q.s. to | q.s. to |
| Injection | 100 ml | 100 ml | 100 ml | 100 ml |

The compositions of Example I to IV were prepared in 300 ml quantities by the following process:

Preparation of Oil Phase: Soya oil was heated to 70-75° C., Monolaurin and Propofol were added and mixed. Egg lecithin was then added into the Soya oil-Propofol mixture and dissolved by stirring.

Preparation of Aqueous Phase: to Water for Injection, Glycerin was added and the pH adjusted to about 10.5 with sodium hydroxide solution.

Emulsification: The Oil Phase was added to the Aqueous Phase with mixing and stirred at high-speed for about 10 minutes to get a coarse emulsion. The coarse emulsion was then homogenized to get desired average globule size of less than 500 nanometers.

The emulsion was filtered, filled in U.S.P. Type I vials and sealed after blanketing with Nitrogen gas. The vials were then sterilized by autoclaving.

### Example V

### Determination of Preservative Efficacy

The compositions of Examples I to IV were tested for preservative efficacy using the following procedure:

Approximately 50-250 colony forming units (cfu)/ml of four standard U.S.P. organisms namely Staphylococcus aureus ATCC 6538, Pseudomonas aeruginosa ATCC 9027, Escherichia coli ATCC 8739 and Candida albicans ATCC 10231; for preservative efficacy tests were inoculated in compositions of each Example and incubated at 32° C.±2° C. The viable count of the test organism was determined after 24 hours.

Method of Determination

### Day 1

1. Inoculate a loopful of axenic (i.e. surgically sterile) culture from a preserved slant into 10 ml of sterile Soyabean-Caesin Broth.
2. Incubate the inoculated tube for 24 hours at 32° C.±2° C.

### Day 2

3. Check for growth and then add 0.2 ml of the culture into 100 ml of sterile Soyabean-Caesin Broth.
4. Incubate the inoculated medium for 18 hours at 32° C.±2° C.

### Day 3

5. After 18 hours of incubation at 32° C.±2° C., check for growth and then transfer 10 ml of the Culture Broth in 15 ml sterile Screw-capped ‘V’ centrifuge tubes.
6. Centrifuge the Culture Broth at 5000 rpm for 10 minutes.
7. Discard the supernatant and re-suspend the pellet in 5 ml sterile saline pH 7.2-7.4, by vortexing the contents for 2 minutes.
8. Centrifuge the contents at 5000 rpm for 10 minutes and discard the supernatant (Wash 1).
9. Re-suspend the pellet in 5 ml sterile saline pH 7.2-7.4, by vortexing the contents for 2 minutes.
10. Centrifuge the contents at 5000 rpm for 10 minutes and discard the supernatant (Wash 2).
11. Again re-suspend the pellet in 5 ml sterile saline pH 7.2-7.4, by vortexing the contents for 3-4 minutes.
12. Prepare a 0.1 O.D.-adjusted cell suspension.
13. Carry out a 7 ten-fold dilution using sterile saline pH 7.2-7.4.
14. Inoculate the test samples (Compositions of Example I to IV) with 0.1 ml of 1:10³ dilution suspension, such that the inoculated samples contain 50-250 cfu/ml
15. Incubate the test samples for 24 hours at 32° C.±2° C.
16. Also surface-spread on media plates 0.1 ml of 1: −10⁴, −10⁵, −10⁶ & −10⁷ dilution. Incubate the plates for 24 hours at 32° C.±2° C. After incubation count the number of colonies on the plates and determine the cell density of 0.1 O.D.-adjusted suspension.

### Day 4

17. After 24-hour incubation, carry out 3 ten-fold serial dilution of the test samples.
18. Surface-spread 0.1 ml of the test samples (undiluted) along with the 3 ten-fold serial dilution tubes onto sterile Soyabean-Caesin Agar Petri plates.
19. Incubate the Petri plates for 24 hours at 32° C.±2° C.

### Day 5

20. Count the number of colonies per plate and determine the cell density.

Observations are provided in Table 2.

TABLE 2

| | S. aureus | | | P. aeruginosa | | | E. coli | | | C. albicans | | |
|---|---|---|---|---|---|---|---|---|---|---|---|---|
| | (cfu/ml) | | | (cfu/ml) | | | (cfu/ml) | | | (cfu/ml) | | |
| | | 24 | 48 | | 24 | 48 | | 24 | 48 | | 24 | 48 |
| Example | Initial | hrs | hrs | Initial | hrs | hrs | Initial | hrs | hrs | Initial | hrs | hrs |
| I | 162 | Nil | Nil | 188 | Nil | Nil | 208 | Nil | Nil | 95 | 209 | 618 |
| II | 162 | Nil | Nil | 188 | Nil | Nil | 208 | Nil | Nil | 95 | 219 | Nil |
| III | 162 | Nil | Nil | 188 | Nil | Nil | 208 | Nil | Nil | 95 | Nil | Nil |
| IV | 162 | Nil | Nil | 188 | Nil | Nil | 208 | Nil | Nil | 95 | 133 | 257 |

Conclusion: Not more than ten-fold increase in the cell counts in the test samples (Compositions of Example I to IV) was observed with Candida albicans at the end of 48 hours and with other organisms bactericidal effect was observed indicating the preservative efficacy of Monolaurin in the compositions.

### Example VI

### 1% Propofol Oil-in-Water Emulsion not Containing Monolaurin (Comparative Example)

The composition was prepared as per Example I except that Propofol quantity is 1 g/100 ml of the composition and Monolaurin is not used.

### Example VII Determination of Preservative Activity

Compositions of Example III and VI were tested for determining preservative activity using the following procedure:

Procedure for Determination of Preservative Efficacy

Approximately 50-250 colony forming units (cfu) per ml of each of Candida albicans ATCC 10231, Pseudomonas aeruginosa ATCC 9027, Escherichia coli ATCC 8739 and Staphylococcus aureus ATCC 6538, the four standard U.S.P. organism cultures specified under “Antimicrobial Effectiveness Testing” were added to a separate aliquot of the product and incubated at 22±2° C. (for fungal cultures) and 32° C.±2° C. (for bacterial cultures). The viable counts of the test organisms were determined after 24 hours and 48 hours.

Method of Determination of Preservative Efficacy for Fungal Culture

### Day 1

1. Inoculate a loopful of axenic culture from a preserved slant into 10 ml of sterile Sabouraud Dextrose Broth.
2. Incubate the inoculated tube for 24 hours at 22±2° C.

### Day 2

3. Check for growth and then add 1.0 ml of the culture into 100 ml of sterile Sabouraud Dextrose Broth.
4. Incubate the inoculated medium for 48 hours at 22° C.±2° C.

### Day 4

5. After 48 hours of incubation at 22° C.±2° C., check for growth and then transfer 10 ml of the Culture Broth in 15 ml sterile Screw-capped ‘V’ centrifuge tubes.
6. Centrifuge the Culture Broth at 5000 rpm for 10 minutes.
7. Discard the supernatant and re-suspend the pellet in 10 ml sterile saline pH 7.2-7.4, by vortexing the contents for 2 minutes.
8. Centrifuge the contents at 5000 rpm for 10 minutes and discard the supernatant (Wash 1).
9. Re-suspend the pellet in 10 ml sterile saline pH 7.2-7.4, by vortexing the contents for 2 minutes.
10. Centrifuge the contents at 5000 rpm for 10 minutes and discard the supernatant (Wash 2).
11. Again re-suspend the pellet in 5 ml sterile saline pH 7.2-7.4, by vortexing the contents for 3-4 minutes.
12. Prepare a cell suspension that gives 50% Optical Transmittance.
13. Carry out a 7 ten-fold dilution using sterile saline pH 7.2-7.4.
14. Inoculate the test samples with 0.1 ml of 1:10³ diluted suspension, such that the inoculated test samples contain 50-250 cfu/ml
15. Incubate the test samples for 24 hours at 22° C.±2° C.
16. Also surface-spread on media plates 0.1 ml of 1:10⁴, 1:10⁵, 1:10⁶ & 1:10⁷ diluted suspension. Incubate the plates for 48 hours at 22° C.±2° C. After incubation count the number of colonies on the plates and determine the cell density inoculated into the test samples.

### Day 5

17. After 24-hour incubation of the test samples, carry out 3 ten-fold serial dilution of the test samples.
18. Surface-spread 0.1 ml of the test samples (undiluted) along with the 3 ten-fold serial dilution tubes onto sterile Sabouraud Dextrose Agar Petri plates.
19. Incubate the Petri plates for 48 hours at 22° C.±2° C.

### Day 7

20. Count the number of colonies per plate and determine the cell density in the test samples (after 24-hour of inoculation).
21. Similarly after 48-hour incubation of the test samples, carry out 3 ten-fold serial dilution of the test samples, surface-spread 0.1 ml of the test samples onto sterile Sabouraud Dextrose Agar Petri plates, incubate for 48 hours at 22° C.±2° C. and determine the cell density in the test samples (after 48-hour of inoculation).

Not more than ten-fold increase in the cell counts in the test samples indicate preservative efficacy of the test samples.

Method of Determination of Preservative Efficacy for Bacterial Cultures

### Day 1

1. Inoculate a loopful of axenic culture from a preserved slant into 10 ml of sterile Soyabean-Caesin Broth.
2. Incubate the inoculated tube for 24 hours at 32° C.±2° C.

### Day 2

3. Check for growth and then add 0.2 ml of the culture into 100 ml of sterile Soyabean-Caesin Broth.
4. Incubate the inoculated medium for 18 hours at 32° C.±2° C.

### Day 3

5. After 18 hours of incubation at 32° C.±2° C., check for growth and then transfer 10 ml of the Culture Broth in 15 ml sterile Screw-capped ‘V’ centrifuge tubes.
6. Centrifuge the Culture Broth at 5000 rpm for 10 minutes.
7. Discard the supernatant and re-suspend the pellet in 5 ml sterile saline pH 7.2-7.4, by vortexing the contents for 2 minutes.
8. Centrifuge the contents at 5000 rpm for 10 minutes and discard the supernatant (Wash 1).
9. Re-suspend the pellet in 5 ml sterile saline pH 7.2-7.4, by vortexing the contents for 2 minutes.
10. Centrifuge the contents at 5000 rpm for 10 minutes and discard the supernatant (Wash 2).
11. Again re-suspend the pellet in 5 ml sterile saline pH 7.2-7.4, by vortexing the contents for 3-4 minutes.
12. Prepare a cell suspension that gives 0.1 Optical Density (O.D.).
13. Carry out a 7 ten-fold dilution using sterile saline pH 7.2-7.4.
14. Inoculate the test samples with 0.1 ml of 1:10³ diluted suspension, such that the inoculated test samples contain 50-250 cfu/ml
15. Incubate the test samples for 24 hours at 32° C.±2° C.
16. Also surface-spread on media plates 0.1 ml of 1:10⁴, 1:10⁵, 1:10⁶ & 1:10⁷ diluted suspension. Incubate the plates for 24 hours at 32° C.±2° C. After incubation count the number of colonies on the plates and determine the cell density inoculated into the test samples.

### Day 4

17. After 24-hour incubation of the test samples, carry out 3 ten-fold serial dilution of the test samples.
18. Surface-spread 0.1 ml of the test samples (undiluted) along with the 3 ten-fold serial dilution tubes onto sterile Soyabean-Caesin Agar Petri plates.
19. Incubate the Petri plates for 24 hours at 32° C.±2° C.

### Day 5

20. Count the number of colonies per plate and determine the cell density in the test samples (after 24-hour of inoculation).
21. Similarly after 48-hour incubation of the test samples, carry out 3 ten-fold serial dilution of the test samples, surface-spread 0.1 ml of the test samples onto sterile Soyabean-Caesin Agar Petri plates, incubate for 24 hours at 32° C.±2° C. and determine the cell density in the test samples (after 48-hour of inoculation).

Not more than ten-fold increase in the cell counts in the test samples indicate preservative efficacy of the test samples.

Results are presented in Table 3-A and 3-B.

The study was carried out in duplicate, indicated in the Table 3-A and 3-B as SET I and SET II.

TABLE 3-A
PRESERVATIVE ACTIVITY OF PROPOFOL EMULSION

| TEST ORGANISMS | | | | | |
|---|---|---|---|---|---|
| S. aureus | | | E. coli | | |
| Zero | 24 | 48 | Zero | 24 | 48 |
| hour | hours | hours | hour | hours | hours |

COMPOSITION OF EXAMPLE VI (COMPARATIVE EXAMPLE WITHOUT MONOLAURIN)

| | | | | | | | |
|---|---|---|---|---|---|---|---|
| LOG. Sub. | SET | 1.81 | 4.02 | NA | 1.80 | 4.06 | NA |
| 10 survivors | I | | | | | | |
| per ml | SET | 2.06 | 4.25 | NA | 1.99 | 4.41 | NA |
| | II | | | | | | |
| Colony forming | SET | 64.0 | 10580.0 | tntc | 64.0 | 11700.0 | tntc |
| units/ml | I | | | | | | |
| (cfu/ml) | SET | 115.0 | 18066.0 | tntc | 99.0 | 26200.0 | tntc |
| | II | | | | | | |
| Fold increase as | SET | NA | 165.0 | tntc | NA | 183.0 | tntc |
| compared to zero | I | | | | | | |
| hour | SET | NA | 157.0 | tntc | NA | 265.0 | tntc |
| | II | | | | | | |

COMPOSITION OF EXAMPLE III (0.01% Monolaurin)

| | | | | | | | |
|---|---|---|---|---|---|---|---|
| LOG. Sub. | SET | 1.80 | 0.00 | NA | 1.80 | 0.00 | NA |
| 10 survivors | I | | | | | | |
| per ml | SET | 2.06 | 0.00 | NA | 1.99 | 0.00 | NA |
| | II | | | | | | |
| Colony forming | SET | 64.0 | 0.0 | 0.00 | 64.0 | 0.0 | NA |
| units/ml | I | | | | | | |
| (cfu/ml) | SET | 115.0 | 0.0 | 0.00 | 99.0 | 0.0 | NA |
| | II | | | | | | |
| Fold increase as | SET | NA | bactericidal | bactericidal | NA | bactericidal | bactericidal |
| compared to zero | I | | | | | | |
| hour | SET | NA | bactericidal | bactericidal | NA | bactericidal | bactericidal |
| | II | | | | | | |

KEY:
tntc—too numerous to count,
NA—Not Applicable

TABLE 3-B
PRESERVATIVE ACTIVITY OF PROPOFOL EMULSION

| TEST ORGANISMS | | | | | |
|---|---|---|---|---|---|
| C. albicans | | | P. aeruginosa | | |
| Zero | 24 | 48 | Zero | 24 | 48 |
| hour | hours | hours | hour | hours | hours |

COMPOSITION OF EXAMPLE VI (COMPARATIVE EXAMPLE WITHOUT MONOLAURIN)

| | | | | | | | |
|---|---|---|---|---|---|---|---|
| LOG. Sub. | SET | 2.44 | 3.85 | 4.80 | 2.03 | 4.45 | NA |
| 10 survivors | I | | | | | | |
| per ml | SET | 2.28 | 3.68 | 4.87 | 2.16 | 4.51 | NA |
| | II | | | | | | |
| Colony forming | SET | 275.0 | 7223.0 | 63100.0 | 107.0 | 28500.0 | tntc |
| units/ml | I | | | | | | |
| (cfu/ml) | SET | 191.0 | 4790.0 | 75000.0 | 145.0 | 32266.0 | tntc |
| | II | | | | | | |
| Fold increase as | SET | NA | 26.0 | 229.0 | NA | 266.0 | tntc |
| compared to zero | I | | | | | | |
| hour | SET | NA | 25.0 | 392.0 | NA | 222.0 | tntc |
| | II | | | | | | |

COMPOSITION OF EXAMPLE III (0.01% Monolaurin)

| | | | | | | | |
|---|---|---|---|---|---|---|---|
| LOG. Sub. | SET | 2.44 | 2.09 | 2.99 | 2.03 | 0.00 | NA |
| 10 survivors | I | | | | | | |
| per ml | SET | 2.28 | 3.03 | 2.96 | 2.16 | 0.00 | NA |
| | II | | | | | | |
| Colony forming | SET | 275.0 | 120.0 | 980.0 | 107.0 | 0.0 | NA |
| units/ml | I | | | | | | |
| (cfu/ml) | SET | 191.0 | 1095.0 | 915.0 | 145.0 | 0.0 | NA |
| | II | | | | | | |
| Fold increase as | SET | NA | 0.4 | 3.5 | NA | bactericidal | bactericidal |
| compared to zero | I | | | | | | |
| Hour | SET | NA | 5.7 | 4.8 | NA | bactericidal | bactericidal |
| | II | | | | | | |

KEY:
tntc—too numerous to count,
NA—Not Applicable

This data in the Table 3-A and 3-B clearly shows that Monolaurin at a concentration of 0.01% w/v is effective in preventing a growth of not more than 10 fold with respect to organisms tested.

With the above observations, it is concluded that monoglyceride is an effective antimicrobial agent to prevent no more than 10-fold growth of susceptible organisms in the compositions as herein described in the text and example and is useful as an antimicrobial agent in a sterile oil-in-water emulsion composition for intravenous administration.

### Example VIII

### Determination of Acute Toxicity

Compositions of Example I and Example VI were subjected to toxicity studies in Swiss Albino Mice.

Twenty healthy Swiss albino mice weighing on an average 20-22 g were used in this study. The animal house of Bharat Serums and Vaccines Ltd. was source of the animals. The animals were isolated for seven days in the quarantine room before use. The animals were given food pellets and tap water ad libitum. The light conditions were 12 hours light and 12 hours dark. The ambient temperature was 22±3° C. The experimental animals were grouped and caged. Colour codes and cages identify the animal numbers. Throughout the study a label would identify each cage with the study number, group number, animal number, sex and details of the treatment.

Acute toxicity was determined after administration of study materials intravenously. All animals were observed for 7 days after administration of study materials for mortality, if any and other clinical signs and symptoms.

Experimental Groups:

Twenty Swiss albino mice were randomly allotted to two groups each comprising of ten animals (Five male & Five female).

Group 1 received single injection of composition of Example VI at a dose of 45 mg/kg intravenously.

Group 2 received single injection of composition of Example I at a dose of 45 mg/kg intravenously.

5% Dextrose Injection was used as a diluent.

Pharmacological Evaluations

Experimental animals were observed for seven days for following parameters:

1. Physical Examination

All the animals were observed after injection every hour to first 4 hours and once daily throughout the experimental period for onset of any changes, degree and duration of changes involving skin, fur, eyes and general behaviour.

2. Mortality

Mortality among the experimental animals was recorded daily throughout the study period up to 7 days.

3. Body Weight:

Each animal was weighed before the administration of study materials and then on 3^{rd} day, 7^{th} day of the study.

4. Statistical Analysis:

Data was recorded, as Mean +SD. Student's paired t-test was used to compare basal and final values of the parametric data.

A ‘p’ value of <0.05 was considered as significant.

5. Observations:

None of the experimental animals showed any adverse signs of toxicity & general behaviour during the study period.

Body weight values of Study Group from Example I were not significantly different from Study Group of Example VI (p>0.05).

LD₅₀ of both compositions of Example I and VI were observed to be more than 45 mg/kg body weight.

There were no any adverse effects observed in both the study group during the study.

An intravenous fat emulsion composition of the present invention is given as Example X. The compositions of comparative Examples IX and XI along with that of Example X are given in Table 4.

TABLE 4
Intravenous Fat Emulsion Compositions

| | | | | | Qty/100 ml | |
|---|---|---|---|---|---|---|
| Examples | IX | | X | | XI | |
| Monolaurin | Nil | | 0.935 | g | Nil | |
| Disodium edetate | Nil | | Nil | | 0.103 | g |
| Soya Oil | 10 | g | 10 | g | 10 | g |
| Egg lecithin | 1.2 | g | 1.2 | g | 1.2 | g |
| Glycerin | 2.25 | g | 2.25 | g | 2.25 | g |
| Sodium hydroxide (0.1 N) | q.s. | | q.s. | | q.s. | |

| | | | |
|---|---|---|---|
| Water for Injection | q.s. to | q.s. to | q.s. to |
| | 100 ml | 100 ml | 100 ml |

The composition of Example IX was prepared by the procedure given below:

Preparation of Oil Phase: Soya oil was heated to 70-75° C.

Preparation of Aqueous Phase: to Water for Injection, Glycerin and Egg lecithin was added and the pH adjusted to about 10.5 with sodium hydroxide solution.

Emulsification: The Oil Phase was added to the Aqueous Phase with mixing and stirred at high-speed for about 10 minutes to get a coarse emulsion. The coarse emulsion was then homogenized to get desired average globule size of less than 500 nanometers.

The emulsion was filtered, filled in U.S.P. Type I vials and sealed after blanketing with Nitrogen gas. The vials were then sterilized by autoclaving.

The composition of Example X was prepared by the following the procedure of Example IX except that Monolaurin was incorporated into the Oil Phase.

The composition of Example XI was prepared by following the procedure of Example IX except that Disodium edetate was incorporated into the Aqueous Phase.

### Example XII

### Safety study with Monolaurin

Compositions of Example IX, X and M were studied along with 5% Dextrose Injection as control.

The objective of this study was to access the safety of Monolaurin as such and in comparison with EDTA when administered into mice by an intravenous route.

Forty healthy Swiss albino mice weighing on an average 20-22 g were used in this study.

Toxicity was determined after administration of study materials. All animals were observed for 7 days after daily administration of study materials for mortality, Clinical signs and symptoms, haematological and histopathological changes.

Data was recorded as Mean +SD and median values for histopathological data.

Student's paired t-test was used to compare basal and final values of the parametric data within the same group and Student's unpaired t-test was used to compare values between two different groups. A ‘p’ value of <0.05 was considered as significant.

Results

All experimental animals were observed daily for seven days for following parameters:

1. Mortality

No deaths observed from any of the experimental groups during the study.

2. Clinical Signs & Pre-Terminal Deaths

No adverse clinical signs & symptoms were observed during the study period.

3. Body Weight

Each animal was weighed before the administration of study material and daily during the treatment period. The data of average weight of each group is provided in Table 5.

TABLE 5
Effect of various compositions (Example IX, X and XI) on animal body weight of mice

| Group | Body weight (gm) | | | | | | | |
|---|---|---|---|---|---|---|---|---|
| (n = 10) | Day 0 | Day 1 | Day 2 | Day 3 | Day 4 | Day 5 | Day 6 | Day 7 |
| Group 1 | 22.21 ± 1.33 | 20.85 ± 1.41 | 21.86 ± 1.88 | 22.2 ± 1.66 | 24.46 ± 2.26 | 24.35 ± 2.16 | 25.06 ± 2.58 | 25.47 ± 2.61 |
| 5% Dextrose Injection | | | | | | | | |
| Group 2 | 21.33 ± 0.77 | 23.00 ± 0.99 | 22.92 ± 1.06 | 23.13 ± 0.93 | 23.7 ± 1.12 | 23.66 ± 1.27 | 24.62 ± 1.65 | 24.56 ± 1.91 |
| Without Monolaurin | | | | | | | | |
| Group 3 | 21.69 ± 1.26 | 22.13 ± 1.25 | 21.82 ± 1.15 | 22.36 ± 1.38 | 23.8 ± 1.55 | 23.5 ± 1.59 | 24.32 ± 1.67 | 23.83 ± 1.89 |
| With Monolaurin | | | | | | | | |
| (1.87 mg/day) | | | | | | | | |
| Group 4 | 21.92 ± 1.10 | 22.63 ± 1.36 | 22.6 ± 1.65 | 22.91 ± 1.97 | 24.13 ± 2.34 | 23.79 ± 2.50 | 23.69 ± 2.83 | 24.32 ± 3.06 |
| With EDTA | | | | | | | | |
| (0.2057 mg/day) | | | | | | | | |

Note:
All figures indicate Mean ± SD.
Group 1: Control
Group 2: Composition of Example IX
Group 3: Composition of Example X
Group 4: Composition of Example XI

5. Haematological Parameters:

The change in final values (Day 7) of while blood cell (WBC), red blood cell (RBC), haemoglobin (HGB), haematocrit (HCT) and platelet (PLT) were not significant (p>0.05) when compared to each other.

6. Organ Weights

Organ wet weights of Liver, Lung, Kidneys, Heart and Spleen in all treatment groups were not significantly different (p>0.05) from each of the groups.

7. Histopathology

Histopathological data of animals from Monolaurin group and EDTA group were observed to compare well.

### Example XIII

The product as per Composition of Example II was prepared and analysed. The methods followed are as follows:
1. Globule size: Globule size is determined based on the principle of measurement of the time-dependent fluctuations of laser-light scattered by particles suspended in solution. BI-90 Plus instrument from Brookhaven Instrument Corporation was used.
2. Propofol and degradation products content: Propofol and degradation products content was determined by HPLC. The details are as follows:

| | |
|---|---|
| Column | Hypersil ODS |
| Detector | Ultraviolet detector |
| Detection wavelength | 270 nm |
| Mobile phase | 60:15:25 Acetonitrile:methanol:10 mM |
| | potassium phosphate Buffer |
| Sample concentration | 0.2 mg/ml |
| Flow rate | 1 ml/min. |

The details on analytical data are provided in Table 6:

TABLE 6
Composition of Example II analytical data:

| Tests | Method | Results |
|---|---|---|
| Appearance | Visual | White opaque liquid |
| Globule size | Photon correlation | |
| Mean 50% | spectroscopy | 0.23 μm |
| High 95% | | 0.38 μm |
| pH | pH Meter | 8.29 |
| Propofol content | HPLC | 10.28 mg/ml |
| Degradation products (as | HPLC | 0.020% |
| % of Propofol): Total | | |
| Bis-propofol | | 0.20% |
| Benzoquinone | | ND |
| Unknown degradants | | ND |
| Free fatty acids | Titrimetric | 4.13 mEq/L |
| Preservative activity | In-house* | Conforms |
| Bacterial endotoxin | U.S.P. | Conforms |
| Sterility | U.S.P. | Sterile |

*Procedure as per Example VII

This data shows that the product prepared complies with the requirements of preservative activity and also other physico-chemical parameters including globule size.

### Example XIV

The prepared composition of Example XIII was subjected to stability studies and the accelerated stability data is provided below in Table 7

TABLE 7
Accelerated stability data at 40° C.

| | Observations | | | |
|---|---|---|---|---|
| Tests | Initial | | 3 month | |
| Appearance | White opaque liquid | | White opaque liquid | |
| pH | 8.29 | | 7.62 | |
| Globule size | | | | |
| Mean 50% | 0.23 | μm | 0.23 | μm |
| High 95% | 0.38 | μm | 0.36 | μm |
| Propofol content | 10.28 | mg/ml | 10.37 | mg/ml |
| Degradation products (as | 0.020% | | 0.052% | |
| % of Propofol): Total | | | | |
| Bis-propofol | 0.020% | | 0.034% | |
| Benzoquinone | ND | | 0.018% | |
| Unknown degradants | ND | | ND | |
| Free fatty acids | 4.13 | mEq/L | 4.95 | mEq/L |

ND = not detected

Composition of Example XIII was subjected to acute toxicity studies as per Example VIII and also preservative activity test as per Example VII initially as well as on completion of 3 month's storage at 40° C. The data obtained indicated comparable toxicity profile and preservative activity as obtained initially.

This data shows that the product is stable and complies with the requirements of satisfactory stability.

### Example XV

### Amikacin Sulphate Emulsion Compositions Containing Preservative Monolaurin

Compositions of Examples XV is given below

| | Qty/100 ml | |
|---|---|---|
| Amikacin sulphate | 6.5 | g |
| Monolaurin | 0.01 | g |
| Soya Oil | 10 | g |
| Egg lecithin | 1.2 | g |
| Glycerin | 2.25 | g |

| | |
|---|---|
| Sodium hydroxide | q.s. |
| (0.1 N) | |
| Water for Injection | q.s. to 100 ml |

The compositions of Example XV was prepared in 300 ml quantities by the following process:

Preparation of Oil Phase: Soya oil was heated to 70-75° C., Monolaurin was added and mixed.

Preparation of Aqueous Phase: to Water for Injection, Glycerin was added and mixed. Egg lecithin was then added to aqueous solution and dispersed by stirring. pH was adjusted to 10.5 with 0.1N sodium hydroxide solution. Weighed quantity of Amikacin sulphate was dissolved in the aqueous phase by stirring.

Emulsification: The Oil Phase was added to the Aqueous Phase with mixing and stirred at high-speed for about 10 minutes to get a coarse emulsion. The coarse emulsion was then homogenized to get desired average globule size of less than 500 nanometers.

The emulsion was filtered, filled in U.S.P. Type I vials and sealed after blanketing with Nitrogen gas. The vials were then sterilized by autoclaving.

### Example XVI

### Propofol Oil-in-Water Emulsion Compositions with Lignocaine Hydrochloride Containing Preservative Monolaurin

Compositions of Examples XVI is given below

| | Qty/100 ml | |
|---|---|---|
| Propofol | 1 | g |
| Monolaurin | 0.01 | g |
| Soya Oil | 10 | g |
| Egg lecithin | 1.2 | g |
| Glycerin | 2.25 | g |
| Lignocaine hydrochloride | 0.25 | g |
| Sodium hydroxide (0.1 N) | q.s. | |
| Water for Injection | q.s. to 100 | ml |

The compositions of Example XVI was prepared in 300 ml quantities by the following process:

Preparation of Oil Phase: Soya oil was heated to 70-75° C., Monolaurin and Propofol were added and mixed. Egg lecithin was then added into the Soya oil-Propofol mixture and dissolved by stirring.

Preparation of Aqueous Phase: to Water for Injection, Glycerin and Lignocaine hydrochloride were added one after the other and mixed well. pH was adjusted to 10.4 with 0.1N sodium hydroxide solution.

Emulsification: The Oil Phase was added to the Aqueous Phase with mixing and stirred at high-speed for about 10 minutes to get a coarse emulsion. The coarse emulsion was then homogenized to get desired average globule size of less than 500 nanometers.

The emulsion was filtered, filled in U.S.P. Type I vials and sealed after blanketing with Nitrogen gas. The vials were then sterilized by autoclaving.

### Example XVII

### Intravenous Fat Emulsion Compositions Containing Preservative Monolaurin

Compositions of Examples XVII is given below

| | Qty/100 ml | |
|---|---|---|
| Monolaurin | 0.01 | g |
| Soya Oil | 5 | g |
| Safflower Oil | 5 | g |
| Egg lecithin | 1.2 | g |
| Glycerin | 2.25 | g |
| Sodium hydroxide | q.s. | |
| (0.1 N) | | |
| Water for Injection | q.s. to 100 | ml |

The compositions of Example XVII was prepared in 300 ml quantities by the following process:

Preparation of Oil Phase: Soya Oil and Safflower Oil were Mixed and heated to 70-75° C., Monolaurin was then added and mixed.

Preparation of Aqueous Phase: to Water for Injection, Glycerin was added and mixed. Egg lecithin was then added to aqueous solution and dispersed by stirring. pH was adjusted to 10.5 with 0.1N sodium hydroxide solution.

Emulsification: The Oil Phase was added to the Aqueous Phase with mixing and stirred at high-speed for about 10 minutes to get a coarse emulsion. The coarse emulsion was then homogenized to get desired average globule size of less than 500 nanometers.

The emulsion was filtered, filled in U.S.P. Type I vials and sealed after blanketing with Nitrogen gas. The vials were then sterilized by autoclaving.

### Example XVIII

### Paclitaxel Oil-in-Water Emulsion Compositions Containing Preservative Monolaurin

Compositions of Examples XVIII is given below

| | Qty/100 ml | |
|---|---|---|
| Paclitaxel | 0.05 | g |
| Monolaurin | 0.01 | g |
| Safflower Oil | 10 | g |
| Egg lecithin | 1.2 | g |
| Glycerin | 2.25 | g |
| Sodium hydroxide (0.1 N) | q.s. | |
| Water for Injection | q.s. to 100 | ml |

The compositions of Example XVIII was prepared in 300 ml quantities by the following process:

Preparation of Oil Phase: Safflower oil was heated to 70-75° C., Monolaurin and Paclitaxel were added and mixed.

Preparation of Aqueous Phase: to Water for Injection, Glycerin was added and mixed well. pH was adjusted to 10.6 with 0.1N sodium hydroxide solution. Egg lecithin was then added and dispersed by stirring.

Emulsification: The Oil Phase was added to the Aqueous Phase with mixing and stirred at high-speed for about 10 minutes to get a coarse emulsion. The coarse emulsion was then homogenized to get desired average globule size of less than 500 nanometers.

The emulsion was filtered, filled in U.S.P. Type I vials and sealed after blanketing with Nitrogen gas. The vials were then sterilized by autoclaving.

### ADVANTAGES OF THE INVENTION

Oil-in-water compositions with Monolaurin do not support microbial growth in case of accidental contamination. Further, compositions containing Monolaurin have been observed to be safe in mice when administered intravenously,

Unlike sodium edetate and sodium pentetate, Monolaurin does not chelate trace metal ions from the biological system and therefore the safety profile is better than the products containing chelating/sequestering agents.

Use of Monolaurin in Propofol compositions is better than use of sulphites because sulphites make the product physically and chemically unstable on long term storage. Sulphites have been reported to support lipid peroxidation in Propofol emulsion and also cause allergic reactions on intravenous administration.

For long term use, benzoates and benzyl alcohol may cause toxicity on intravenous administration whereas Monolaurin can be used without induction of any toxicity (for over 7 days).

The products of present invention give a Propofol oil-in-water emulsion composition that can be used for intravenous administration for prolonged period required clinically without the fear of microbial contamination and infection.

The oil-in-water intravenous fat emulsion compositions of present invention can be used as a total parenteral nutrition mixture for a prolonged period without the fear of microbial contamination and infection.

Oil-in-water emulsion compositions of present invention containing hydrophilic/and lipophilic drugs can be used for a prolonged period without the fear of microbial contamination and infection.

Similarly the oil-in-water emulsion compositions of the present invention comprising intravenous fat emulsion composition and hydrophilic/lipophilic drugs can also be used for a prolonged period without the fear of microbial contamination and infection.

The present invention also provides intravenous administrable tailor made compositions having both drug and nutritional components as required by the patient.

It will be understood that the invention is not restricted to the specific details described above but that numerous modifications and variations can be made without departing from the invention as defined by the following claims.

1. A sterile oil-in-water emulsion composition for intravenous administration comprising, as an antimicrobial preservative, a monoglyceride.

2. An intravenously administrable composition as claimed in claim 1, wherein the monoglyceride is present in an amount sufficient to prevent a no more than 10 fold increase in the growth of microbial cultures each of caqdida altzcczs ATCC 10231, Pseudomonas aerugzroscz ATCC 9027, escherichia colt ATCC S739 and staphylococcus agrees ATCC 6538 for at least 24 hours as measured by a test wherein a washed suspension of each organism is added to a separate aliquot of said composition at approximately 50 colony forming units per-ml- and incubated at al temperature in the range of 20-25° C. for culture of candida albicans and at a temperature in the range of 30-35° C. for the remaining cultures and are tested for viable counts of said organisms after 24 hours and wherein the said amount of monoglyceride is no more than the antimicrobial equivalent against said cultures obtained with a composition containing 1.5% w/v Monolaurin.

3. An intravenously administrable composition as claimed in claim 1, wherein the monoglyceride is monolaurin.

4. An intravenously administrable composition as claimed in claim 1, wherein said amount of monoglyceride is the antimicrobial equivalent against said cultures obtained with a composition containing up to 1% w/v monolaurin.

5. An intravenously administrable composition as claimed in claim 4, wherein said amount of monoglyceride is the antimicrobial equivalent against said cultures obtained with a composition containing up to 0.5% w/v monolaurin.

6. An intravenously administrable composition as claimed in claim 5, wherein said amount of monoglyceride is the antimicrobial equivalent against said cultures obtained with a composition containing up to 0.1% w/v monolaurin.

7. An intravenously administrable composition as claimed in claim 1, wherein said composition is for total parenteral nutrition.

8. An intravenously administrable composition as claimed in claims 1, wherein said composition is a medicament comprising a lipophilic drug.

9. An intravenously administrable composition as claimed in claim 8, wherein the lipophilic drug is propofol.

10. An intravenously administrable composition as claimed in claim 8 or claim 9, wherein the content of lipophilic drug is from 0.01% w/v to 5% w/v of the composition.

11. An intravenously administrable composition as claimed in claim 10, wherein the content of lipophilic drug is from 0.1% to 2% w/v of the composition.

12. An intravenously administrable composition as claimed in claim 8, wherein the ratio of monoglyceride (calculated as monolaurin) to lipophilic drug is from 1:0.01 to 1:5000 by weight.

13. An intravenously administrable composition as claimed in claim 12, wherein the ratio of monoglyceride (calculated as monolaurin) to lipophilic drug is from 1:0.2 to 1:1000 by weight.

14. An intravenously administrable composition as claimed in claim 13, wherein the ratio of monoglyceride (calculated as monolaurin) to lipophilic drug is from 1:4 to 1:200 by weight.

15. An intravenously administrable composition as claimed in claim 14, wherein the ratio of monoglyceride (calculated as monolaurin) to lipophilic drug is from 1:20 to 1:100 by weight.

16. An intravenously administrable composition as claimed in claim 1, comprising at least one triglyceride oil and at least one phosphatide.

17. An intravenously administrable composition as claimed in claim 16, wherein the at least one triglyceride oil is selected from natural vegetable oils and synthetic MCT (medium-chain triglycerides) oil.

18. An intravenously administrable composition as claimed in claim 16, wherein the content of said triglyceride gilts) is not more than 30% w/v of the composition.

19. An intravenously administrable composition as claimed in claim 18, wherein the content of said triglyceride oil(s) is from 5% w/v to 20% w/v of the composition.

20. An intravenously administrable composition as claimed in claim 16, wherein the at least one phosphatide is selected from purified egg lecithin and purified soya lecithin.

21. An intravenously administrable composition as claimed in claim 16, wherein the content of the phosphatide(s) is from 0.1% w/v to 3% w/v of the composition.

22. An intravenously administrable composition as claimed in claim 1 comprising at least one isotonic agent.

23. An intravenously administrable composition as claimed in claim 22, wherein the at least one isotonic agent is glycerin.

24. An intravenously administrable composition as claimed in claim 1, wherein said oil phase comprises lipophilic drugs and/or nutritive oils, and aqueous phase comprises hydrophilic drugs and/or water soluble nutrients.

25. An intravenously administrable composition as claimed in claim 1, wherein the pH of the composition is between 6 and 8.5.

26. An intravenously administrate composition as claimed in claim 9 comprising propofol about 1% w/v, Soybean oil about 10% w/v, purified egg lecithin about 1.2% w/v, glycerin about 2.25% w/v, monolaurin about 0.05% w/v, sodium hydroxide sufficient to bring the pH between 6 and 8.5 and water for injection to make up to 100% by volume.

27. An intravenously administrable composition as claimed in claim 9 comprising propofol about 2% w/v, soybean oil about 10% w/v, purified egg lecithin about 1.2% w/v, glycerin about 2.25% w/v, monolaurin about 0.05% w/v, sodium hydroxide sufficient to bring the pH between 6 and 8.5 and water for injection to make up to 100% by volume.

28. An intravenously administrable composition as claimed in claim 9 comprising propofol about 1% w/v, soybean oil about 10% w/v, purified egg lecithin about 1.2% w/v, glycerin about 2.25% w/v, monolaurin about 0.01% w/v, sodium hydroxide sufficient to bring the pH between 6 and 8.5 and water for injection to make up to 100% by volume.

29. A method of intravenously administering an oil-in-water emulsion composition comprising, as an antimicrobial preservative, a monoglyceride.

30. The method of claim 29 wherein the components of the intravenous administration composition are as defined in claim 26.

31. A process of preparing an intravenously administrable composition as claimed in claim 8 comprising the steps of: i) dissolving monoglyceride and the lipophilic drug in triglyceride oil maintained at elevated temperature; ii) adding and dissolving phosphatide in the solution prepared in step i); iii) preparing an aqueous phase by dissolving glycerin and sodium hydroxide in water and then heating the aqueous phase; iv) adding the solution of step ii) to the aqueous phase obtained at step iii) under stirring to produce a coarse emulsion; and v) homogenizing the coarse emulsion obtained at step iv).

32. A process of preparing an intravenously administrable composition as claimed in claim 8 comprising the steps of: i) dissolving monoglyceride and, if present, the lipophilic drug in triglyceride oil maintained at elevated temperature; ii) preparing an aqueous phase by dissolving glycerin and sodium hydroxide in water and then heating the aqueous phase; iii) adding and dispersing phosphatide in the aqueous phase prepared in step ii); iv) adding the solution of step i) to the aqueous phase obtained at step iii) under stirring to produce a coarse emulsion; and v) homogenizing the coarse emulsion obtained at step iv).

33. A process as claimed in claim 32, wherein said homogenization is to an average globule size of less than SOO nanometers; the homogenized composition is Altered; the resultant filtrate is filled into containers, followed by nitrogen blanketing and the filled containers sealed; and the sealed containers filled with the said filtrate sterilized by autoclaving.

34. An intravenously administrable composition as claimed in claim 1, wherein the aqueous phase comprises hydrophilic drugs and/or water soluble nutrients.

35. (canceled)
